(19) United States
(12) Patent Application Publication
(10) Pub. No.: US 2009/0041750 A1
(43) Pub. Date: Feb. 12, 2009

(54) Treatment of spongiform encephalopathy by increased hydrogen peroxide degradation

(76) Inventor: Ivan Petyaev, Cambridge (GB)

Correspondence Address: NIXON & VANDERHYE, PC, 901 NORTH GLEBE ROAD, 11TH FLOOR, ARLINGTON, VA, 22203, US

(21) Appl. No.: 11/663,554

(22) PCT Filed: Sep. 23, 2005
(86) PCT No.: PCT/GB2005/003681

§ 371 Date: Feb. 14, 2008

(30) Foreign Application Priority Data
Sep. 23, 2004 (GB) 0421244.5

(51) Int. Cl. A61K 39/395; C12Q 1/30; C12Q 1/28; A61K 38/02; A61P 25/00; G01N 33/68; A01K 67/027

(52) U.S. Cl. 424/130.1; 435/27; 435/28; 800/3; 800/9; 800/21; 800/25; 800/13; 514/2

(57) ABSTRACT

The present invention relates to the treatment of CJD and other spongiform encephalopathies by increasing the level and/or activity of hydrogen peroxide degrading enzymes, such as catalase and glutathione peroxidase, in order to deplete hydrogen peroxide and prevent the build up of gaseous oxygen in the brains of patients. Various aspects are provided, including screening methods for agents for the treatment of spongiform encephalopathy.

This invention relates to the treatment of spongiform encephalopathy and the provision of therapeutic agents for use in such treatment.

Spongiform encephalopathies are a class of chronic progressive degenerative conditions affecting the central nervous system. There is no treatment, and the conditions are invariably fatal.

Spongiform degeneration is the major distinguishing feature of a spongiform encephalopathy. However, its cause, in sporadic cases, and pathogenesis remain unclear. Some spongiform encephalopathies are transmissible and are caused by a prion agent. A prion is an aberrant form of a normal protein that causes the normal protein to conform to its aberrant shape, leading to a cascade of abnormal proteins accumulating in brain cells. This accumulation produces holes or cavities within the brain, giving a “sponge-like” appearance.

Experiments in vitro¹, on cell cultures² and on animal models³ have shown that a pathogenic form of prion alters the activities of antioxidant enzymes. This may stimulate processes of oxidative degradation in the affected human brain. Analysis of cerebrospinal fluid of patients with sporadic and familiar CJD provides indirect indication that processes of lipid peroxidation can be activated in the central nervous system⁴. However, there have been no direct studies of oxidative processes on the brains of patients with CJD.

The present inventor has identified key defects in the oxidative systems within the brains of CJD sufferers. In particular, the brains of CJD patients show a significant reduction in the activity of hydrogen peroxide degrading enzymes. This reduction in activity leads to the build up of hydrogen peroxide, which breaks down spontaneously to produce gaseous oxygen. The build up of gaseous oxygen in the brains of CJD patients leads to the formation of the sponge-like cavities within the brain that are characteristic of CJD.

The present invention, in various aspects, relates to methods and means for depleting levels of hydrogen peroxide in the brain by increasing the level and/or activity of hydrogen peroxide degrading enzymes, thereby reducing the production of gaseous oxygen.

An aspect of the invention provides a method for identifying and/or obtaining an agent for the treatment of spongiform encephalopathy comprising;

i) contacting a hydrogen peroxide degrading enzyme with a test compound, and,
ii) determining the activity of the hydrogen peroxide degrading enzyme activity.

Hydrogen peroxide degrading enzymes include glutathione peroxidase and catalase.

Glutathione peroxidase (EC1.11.1.9) catalyses the reaction:

2 glutathione+H₂O₂ [symbol] 2H₂O+oxidized glutathione

Glutathione peroxidase may be a mammalian glutathione peroxidase, in particular a human glutathione peroxidase. Human glutathione peroxidases are well known in the art and include for example glutathione peroxidase 1 (gi|32363469; AAP80181.1), glutathione peroxidase 2 (gi|45501022; AAH67221.1), glutathione peroxidase 3 (gi|31559182; AAP50261.1), glutathione peroxidase 4 (gi|34782963; AAH11836.1), glutathione peroxidase 5 (gi|3288455; CAA06463.1), glutathione peroxidase 6 (gi|32492913; AAP85543.1) and glutathione peroxidase 7 (gi|21595679; AAH32788.1).

Catalase (EC1.11.1.6) catalyses the reaction:

2H₂O₂ [symbol] 2H₂O+O₂

Catalase may be a mammalian catalase, in particular human catalase (Database Acc No: P04040, GI: 115702).

The activity of a hydrogen peroxide degrading enzyme in the presence of a test compound may be compared with activity in comparable reaction medium and conditions in the absence of a test compound. An increase in activity in the presence of test compound relative to the absence is indicative that the test compound may be useful in the treatment of spongiform encephalopathy.

A spongiform encephalopathy may be a transmissible spongiform encephalopathy (TSE). A spongiform encephalopathy may include Creutzfeldt-Jakob disease (CJD), Kuru, Gerstmann-Straussler-Scheiker syndrome, scrapies and BSE. CJD may include sporadic, familial, latrogenic and variant CJD.

The determination of hydrogen peroxide degrading enzyme activity may be quantitative or qualitative and may include detecting the existence of the activity, which may, for example, include detecting the existence of activity above a certain threshold value, and measuring the amount or level of the activity.

In some embodiments, hydrogen peroxide degrading enzyme activity may be determined prior to introduction of the test compound to obtain a baseline value and changes in hydrogen peroxide degrading enzyme activity in the presence of the compound determined.

A suitable hydrogen peroxide degrading enzyme polypeptide may be isolated or may be contained or comprised within a cell.

For example, a method for identifying and/or obtaining an agent for the treatment of spongiform encephalopathy may comprise;
i) contacting a cell with a test compound, and
ii) determining the hydrogen peroxide degrading enzyme activity of said cell.

Suitable cells for use in the present methods are mammalian cells, preferably human cells. In preferred embodiments, the cell may be a neural cell, for example a CNS cell, including a brain cell.

A suitable cell may have endogenous hydrogen peroxide degrading enzyme activity (i.e. it may naturally express a hydrogen peroxide degrading enzyme polypeptide) or may be engineered to express a hydrogen peroxide degrading enzyme polypeptide, for example a heterologous hydrogen peroxide degrading enzyme polypeptide encoded by a nucleic acid introduced into the cell by recombinant technology or an endogenous hydrogen peroxide degrading enzyme polypeptide activated by gene activation technology.

Hydrogen peroxide degrading enzyme activity may be determined by measuring the accumulation of products or by-products, such as coupled reporter molecules or the disappearance or consumption of substrates such as hydrogen peroxide.

Suitable methods are, for example, described in CRC Handbook of Methods for Oxygen Radical Research, CRC Press, Boca Raton, Fla. (1985), Oxygen Radicals in Biological Systems. Methods in Enzymology, v. 186, Academic Press, London (1990); Oxygen Radicals in Biological Systems. Methods in Enzymology, v. 234, Academic Press, San Diego, New York, Boston, London (1994); and Free Radicals. A practical approach. IRL Press, Oxford, New York, Tokyo (1996).

Many methods for determining hydrogen peroxide degrading enzyme activity are known in the art and are suitable for use in accordance with the present invention. The precise mode of determining hydrogen peroxide degrading enzyme activity is not a feature of the present invention and those skilled in the art are able to choose a suitable mode according to their preference and general knowledge.

The test compound may enhance or increase the activity and/or stability of hydrogen peroxide degrading enzyme or the expression of hydrogen peroxide degrading enzyme. In some embodiments, the compound may have hydrogen peroxide degrading enzyme activity itself i.e. it may catalyse the breakdown of hydrogen peroxide into oxygen and water.

In order to act directly on the brain, compounds useful in the treatment of spongiform encephalopathy are preferably able to cross the blood brain barrier (BBB).

A method as described herein may further comprise determining the ability of a test compound to cross the BBB.

This may be achieved for example by introducing the compound to a test animal, for example orally or parenterally (e.g. intravenously), and determining the amount of compound which enters the brain of the animal.

In some embodiments, the test compound may be a compound of a type which is known to cross the BBB, such as a small molecule which is fat-soluble.

In other embodiments, the ability of a test compound to cross the BBB may be determined after determining its effect on hydrogen peroxide degrading enzyme expression and/or activity i.e. a compound active in increasing hydrogen peroxide degrading enzyme expression and/or activity may be tested for ability to cross the BBB.

Test compounds that show little or no ability to cross the BBB may be modified in order to increase this activity. Modification of compounds is described in more detail below.

In some embodiments, a primary screen may comprise determining the ability of a compound to cross the BBB in a test animal and determining the ability of the compound to increase hydrogen peroxide degrading enzyme activity.

A method of identifying a test compound as an agent for use in the treatment of spongiform encephalopathy may comprise:

(a) introducing a test compound into a non-human animal; and
(b) determining the level and/or activity of hydrogen peroxide degrading enzyme in the brain of said animal.

The compound may be introduced parenterally into the animal, for example intravenously.

A non-human animal is preferably a mammal, for example a rodent such as a rat or mouse, or an agricultural animal such as a sheep or cow.

The non-human animal may be a model for a spongiform encephalopathy. For example, the animal may have a transmissible spongiform encephalopathy (TSE), for example a scrapies infected sheep or a BSE infected cow, or may have genetic or other defects that model the symptoms of spongiform encephalopathy. In such embodiments, alternatively or additionally to the activity and/or level of hydrogen peroxide degrading enzyme, a biological parameter such as prion deposition or cavitation may determined in the brain of the animal model or other neurological symptoms of the TSE may be assessed.

An increase in the activity and/or level of hydrogen peroxide degrading enzyme in the brain of an animal to whom the test compound is administered, for example an increase in the cerebellum or cerebral cortex or other area of the brain of the animal, relative to the brain of a control animal, is indicative that the test compound both affects hydrogen peroxide degrading enzyme expression and/or activity and crosses the BBB and may therefore be useful in the treatment of spongiform encephalopathy.

In some embodiments, the test compound introduced into the animal is a compound identified as increasing the expression and/or activity of hydrogen peroxide degrading enzyme in an in vitro or cellular screen as described above.

In some embodiments, methods as described above may comprise the further step of sacrificing the non-human animal.

Another aspect of the invention provides a non-human animal model for use in screening for compounds useful in the treatment of spongiform encephalopathy, the animal having reduced or deficient levels and/or activity of a hydrogen peroxide degrading enzyme, such as catalase and/or glutathione peroxidase.

Such an animal may display one or more symptoms of spongiform encephalopathy. In some embodiments, the animal may exhibit a spongiform encephalopathy phenotype.

A test animal for use in screening for compounds useful in the treatment of spongiform encephalopathy may be produced by;
reducing or abrogating the expression and/or activity of a hydrogen peroxide degrading enzyme in an animal.

Methods of reducing or abrogating the expression of particular genes in an animal model (e.g. producing ‘knock out’ animals) are well known in the art and are described in more detail below.

Preferred animal models for use in screening methods include rodents, such as rats or mice.

A related aspect provides the use of an animal having reduced or deficient levels and/or activity of hydrogen peroxide degrading enzyme in a method of screening for a compound which is useful in the treatment of spongiform encephalopathy.

Methods of screening for a compound useful in the treatment of spongiform encephalopathy are described in more detail above.

The precise format of assay or screening methods may be varied by those of skill in the art using routine skill and knowledge. In particular, high throughput formats may be useful for large scale automated screening programs.

A test compound suitable for use in the present methods may be a small chemical entity, peptide, antibody molecule or other molecule whose effect on the hydrogen peroxide degrading enzyme activity is to be determined. Suitable test compounds may be selected from compound collections and designed compounds, for example using combinatorial chemistry as described below.

Combinatorial library technology (Schultz, J S (1996) Biotechnol. Prog. 12:729-743) provides an efficient way of testing a potentially vast number of different substances for ability to modulate the activity of a hydrogen peroxide degrading enzyme. As described above, prior to or as well as being screened for modulation of hydrogen peroxide degrading enzyme activity, test substances may be screened for ability to bind with the hydrogen peroxide degrading enzyme. This may be used as a coarse screen prior to testing a substance for actual ability to increase or enhance the activity of hydrogen peroxide degrading enzyme.

The amount of test substance or compound which may be added to a method described herein will normally be determined by trial and error depending upon the type of compound used. Typically, from about 0.01 to 100 nM concentrations of putative inhibitor compound may be used, for example from 0.1 to 10 nM. Compounds which may be used may be natural or synthetic chemical compounds used in drug screening programmes. Extracts of plants which contain several characterised or uncharacterised components may also be used.

Other candidate compounds may be based on modelling the 3-dimensional structure of the hydrogen peroxide degrading enzyme and using rational drug design to provide potential enhancer compounds with particular molecular shape, size and charge characteristics.

In some embodiments, test compounds may be small fat-soluble molecules or other molecules which cross the BBB.

The effect on one or more biological functions of a compound identified by a method described above may be assessed in a secondary screen. Suitable biological functions which may be assessed in a secondary screen include delay, reduction or amelioration of the symptoms or effects of a spongiform encephalopathy. Typical symptoms of spongiform encephalopathy are well known in the art.

A method may comprise identifying a test compound as an agent which modulates the expression and/or activity of a hydrogen peroxide degrading enzyme in said cell.

A test compound identified as an agent which increases the expression and/or activity of hydrogen peroxide degrading enzyme in said cell may be isolated and/or purified.

In some embodiments, a compound may be prepared, synthesised and/or manufactured using conventional synthetic techniques.

Optionally, compounds identified as agents which increase the expression and/or activity of a hydrogen peroxide degrading enzyme using an assay method described herein may be modified to optimise activity or provide other beneficial characteristics such as increased half-life, reduced side effects upon administration to an individual or improved passage across the BBB.

The modification of a known pharmacologically active compound to improve its pharmaceutical properties is a known approach to the development of pharmaceuticals based on a “lead” compound. This might be desirable where the active compound is difficult or expensive to synthesise or where it is unsuitable for a particular method of administration, e.g. peptides are not well suited as active agents for oral compositions as they tend to be quickly degraded by proteases in the alimentary canal. The design, synthesis and testing of modified active compounds, including mimetics, may be used to avoid randomly screening large number of molecules for a target property.

There are several steps commonly taken in modifying a compound which has a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled to according its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of hydrogen peroxide degrading enzyme is modelled. This can be especially useful where the ligand and/or binding partner change conformation on binding, allowing the model to take account of this the design of the mimetic.

A template molecule is then selected onto which chemical groups which mimic the pharmacophore can be grafted. The template molecule and the chemical groups grafted on to it can conveniently be selected so that the modified compound is easy to synthesise, is likely to be pharmacologically acceptable, and does not degrade in vivo, while retaining the biological activity of the lead compound. Modified compounds found by this approach can then be screened to see whether they have the target property, or to what extent they exhibit it.

In some embodiments, a compound may be modified to improve or increase its ability to enter the brain across the BBB. For example the test compound may be lipidized or conjugated to a transport molecule that crosses the BBB. Conjugation of a compound to a transport molecule may be direct or indirect, covalent, e.g. via a peptide bond, or non-covalent. Linkage via a peptide bond may be as a result of recombinant expression of a gene fusion encoding transport molecule (e.g. an antibody) and the test compound.

Further optimisation or modification can then be carried out to arrive at one or more final compounds for in vivo or clinical testing.

The test compound may be formulated into a composition, such as a medicament, pharmaceutical composition or drug, with a pharmaceutically acceptable excipient as described below.

A method of producing a pharmaceutical composition for use in treating spongiform encephalopathy may comprise;
identifying a compound which increases the expression and/or activity of hydrogen peroxide degrading enzyme using a method described herein; and,
admixing the compound identified thereby with a pharmaceutically acceptable carrier.

As described above, the compound may be modified to optimise the pharmaceutical properties thereof.

A method for preparing a pharmaceutical composition for the treatment of a spongiform encephalopathy may comprise;

i) identifying a compound which increases the expression and/or activity of hydrogen peroxide degrading enzyme in a cell, for example using a method as described herein.
ii) synthesising the identified compound, and;
iii) incorporating the compound into a pharmaceutical composition.

A pharmaceutical composition may comprise a test compound identified as a stimulator or enhancer of hydrogen peroxide degrading enzyme expression and/or activity.

The present invention encompasses a compound identified using a method described above as an agent which may be useful in the treatment of spongiform encephalopathy, a pharmaceutical or veterinary composition, medicament, drug or other composition comprising such a compound, a method comprising administration of such a composition to a patient, e.g. for treatment (which may include preventative treatment) of a spongiform encephalopathy, use of such a compound in manufacture of a composition for administration, e.g. for treatment of a spongiform encephalopathy, and a method of making a pharmaceutical or veterinary composition comprising admixing such a compound with a pharmaceutically acceptable excipient, vehicle or carrier, and optionally other ingredients.

A compound identified by a method described herein may be administered to a patient for treatment of a spongiform encephalopathy.

A compound may be administered in combination with other therapeutic agents or treatments. For example, a compound may be administered in combination with hyperbaric treatment i.e. subjecting the individual to recompression therapy to dissolve any gaseous oxygen present in the brain and allow it to diffuse out of the brain.

Another aspect of the invention provides a method of treating a spongiform encephalopathy in an individual comprising;

increasing the expression and/or activity of hydrogen peroxide degrading enzyme in the brain of the individual.

The expression and/or activity of hydrogen peroxide degrading enzyme may for example, be increased by administration of a compound identified by a method described herein. A compound may possess hydrogen peroxide degrading activity or it may be a co-factor or agonist which increases the activity of endogenous hydrogen peroxide degrading enzyme or it may increase the expression of endogenous hydrogen peroxide degrading enzyme.

An individual suitable for treatment by the present methods may include any mammal, in particular domestic or agricultural animals such as sheep and cows. In preferred embodiments, the individual is human.

An individual suitable for treatment in accordance with the present methods may be suffering from a spongiform encephalopathy, may be suspected of suffering from a spongiform encephalopathy, or may be susceptible to spongiform encephalopathy. Although confirmation of a diagnosis of spongiform encephalopathy in an individual generally requires post-mortem analysis of the brain, individuals suitable for treatment in accordance with the present methods may be identified using established clinical criteria.

Whether it is a polypeptide, peptide, nucleic acid molecule, small molecule or other pharmaceutically useful compound that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

A composition may be administered alone or in combination with other treatments, either simultaneously or sequentially dependent upon the condition to be treated.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, or Lactated Ringer's Injection. Preservatives, stabilisers, buffers, antioxidants and/or other additives may be included, as required.

Other aspects of the invention relate to determining and/or reducing the susceptibility of animals to a spongiform encephalopathy.

A method of determining the susceptibility of an animal to a spongiform encephalopathy may comprise;
determining the level or activity of hydrogen peroxide degrading enzyme in a sample obtained from said animal.

A sample may be any tissue sample from the animal, for example nervous tissue, in particular brain tissue.

The level or activity of hydrogen peroxide degrading enzyme may be determined by any suitable technique. Examples of suitable techniques are described herein.

The susceptibility of any animal may be determined, for example a non-human animal such as a cat, cattle, dog, horse, goat, rodent, and sheep. In some embodiments, an agricultural animal such as sheep or cattle may be used.

The susceptibility of an animal or its progeny to spongiform encephalopathy may be modulated by altering the level or amount of hydrogen peroxide degrading activity in the tissue of the animal, in particular in the brain of the animal.

For example, the susceptibility of an animal to a spongiform encephalopathy may be reduced by;
increasing the expression and/or activity of hydrogen peroxide degrading enzyme in the brain of said animal.

The expression and/or activity of hydrogen peroxide degrading enzyme may be increased using recombinant techniques. For example, a heterologous nucleic acid encoding a hydrogen peroxide degrading enzyme may be introduced into one or more cells of an animal and expressed.

A method of generating an animal having reduced spongiform encephalopathy susceptibility may comprise:
introducing a recombinant nucleic acid encoding a hydrogen peroxide degrading enzyme into one or more cells of said animal.

The expression of the hydrogen peroxide degrading enzyme in tissue of the animal, in particular the brain, may be determined following introduction of the nucleic acid into the animal or an ancestor of the animal.

A method of producing an animal having reduced spongiform encephalopathy susceptibility may comprise:

a) introducing a nucleic acid encoding a hydrogen peroxide degrading enzyme into a fertilized egg or an embryo of an animal;
b) transferring the egg or embryo comprising the nucleic acid to a surrogate mother animal; and
c) allowing the transferred egg or embryo comprising the nucleic acid to develop to term to produce a transgenic animal whose genome comprises a nucleic acid encoding an hydrogen peroxide degrading enzyme polypeptide.

Expression of the nucleic acid results in the transgenic animal exhibiting a phenotype of reduced susceptibility to spongiform encephalopathy.

The nucleic acid may comprise a regulatory sequence, for example a nervous tissue specific regulatory sequence, operably linked to the nucleic acid sequence encoding the hydrogen peroxide degrading enzyme.

The animal may be bred to provide progeny of the transgenic animal. The genome of the progeny will comprise a heterologous nucleic acid encoding a hydrogen peroxide degrading enzyme, as described above, expression of which results in the transgenic animal exhibiting a phenotype of reduced susceptibility to spongiform encephalopathy.

A heterologous nucleic acid is a nucleic acid sequence that has been introduced into cells or ancestors thereof, using genetic engineering, i.e. by human intervention. Nucleic acid heterologous, or exogenous or foreign, to a cell may be non-naturally occurring in cells of that type, variety or species. A nucleic acid sequence within a host cell may be identifiably heterologous, exogenous or foreign.

The transgenic or somatic recombinant non-human animals may be generated according to well established methods for introduction of a recombinant DNA construct allowing germ-line or somatic insertion including viral or non-viral vector-mediated gene transfer into fertilized eggs, zygotes or early embryos and/or a specific tissue (such as brain) in the adult animal, e.g. by gene transfer into embryonic stem cells, retroviral infection of early embryos or pronuclear microinjection. Further manipulation of resulting fertilized eggs, zygotes or early embryos and breeding of resulting transgenic founder animals follows established routes of breeding transgenic animals. Transgenic cells expressing heterologous hydrogen peroxide degrading enzyme may be prepared by any technique known in the art, for example the recombinant DNA construct may be introduced by direct DNA microinjection, DNA transfection, viral or non-viral vectors, or the cells may be obtained from transgenic or somatic recombinant non-human animals, and cultured in vitro.

In other embodiments, the expression and/or activity of hydrogen peroxide degrading enzyme may be increased using non-recombinant techniques. For example, a selective breeding program may be used to produce animals which possess high levels or activity of endogenous hydrogen peroxide degrading enzymes.

A method for determining the inclusion of an animal in a breeding program to reduce the susceptibility to spongiform encephalopathy in progeny, may comprise:
determining the level or activity of hydrogen peroxide degrading enzyme in a tissue sample from the animal,
enhanced or increased activity or levels being indicative of an inheritable trait of reduced susceptibility to spongiform encephalopathy; and,
selecting for inclusion in said breeding program animals that exhibit enhanced or increased levels of hydrogen peroxide degrading enzymes.

Methods of determining the level or activity of hydrogen peroxide degrading enzyme in a tissue sample from the animal are described above.

The selective breeding of animals which exhibit increased levels of hydrogen peroxide degrading enzymes leads to the progressive stable increases in levels of hydrogen peroxide degrading enzymes in progeny. The operation of selective breeding programs are well known in the art.

Another aspect of the invention provides a non-human animal having reduced susceptibility to spongiform encephalopathy which is obtainable or obtained by a method described above. Such an animal may have increased or enhanced levels or activity of hydrogen peroxide degrading enzymes relative to control animals.

Aspects of the present invention will now be illustrated with reference to the accompanying table described below and experimental exemplification, by way of example and not limitation. Further aspects and embodiments will be apparent to those of ordinary skill in the art.

All documents mentioned in this specification are hereby incorporated herein by reference.

Table 1 shows superoxide dismutase, peroxidase and catalase activity in the grey matter of the brain of the patient with NVCJD. Enzymatic activities are expressed: for SOD as a reverse level of diformazan staining in riboflavin auto-oxidised system, for peroxidase as a direct level of the density of o-dianisidine oxidised by H₂O₂, for catalase in μM H₂O₂/min/ml.

### EXAMPLES

Two major antioxidant enzymes, superoxide dismutase (SOD) and catalase, were tested in the cerebellum and cortex of a patient who had died from new variant Creutzfeld-Jakob disease (NvCJD). An achromatic staining pattern for SOD showed that this activity is present mainly in the grey matter, and in particular in neuron cytoplasm, of both the cerebral and cerebellar cortex. Comparison of these samples with similar ones from the brain of a healthy person, who died in a traffic accident, and a person, who died from complications of motor neuron disease, revealed a significant loss of SOD activity in the brain of the patient with NvCJD. There was a 2-fold reduction in the cerebellar cortex and 6-fold reduction in cerebral cortex (table 1).

Measurement of catalase registered more profound changes. The decrease in activity of the enzyme was 3-fold in cerebellum, but in the cerebral cortex it was reduced to an undetectable level (table 1). The depression of catalase activity leads to an excessive accumulation of hydrogen peroxide, which itself is a powerful oxidant in biological systems. It can also be used by SOD as its substrate. As a result of this, one of the main antioxidant enzymes can be converted into its opposite pro-oxidant form, which would be capable of oxidising and damaging biologically important molecules and cellular membranes⁵⁻⁷.

Direct staining for peroxidase activity of SOD shows strong activity in both cerebral and cerebellar cortices taken from the patient with NvCJD, but no activity was detectable in the control (table 1). A specific inhibitor of SOD, diethyldithiocarbamate, completely abolished the peroxidase activity of the enzyme.

In NvCJD loss of antioxidant enzymatic defence and appearance of measurable SOD-peroxidase activity was observed in brain areas that show spongiform degeneration of neurons and accumulation of abnormal prions, namely cerebral and cerebellar cortex⁸.

### REFERENCES

1. Brown, D. R. Biochem. J., 352, 511-518 (2000).
2. Milhavet, O. et al. Proc Natl. Acad. Sci. USA, 97, 13937-13942 (2000).
3. Lee, D. W. et al. Free Radic. Res., 30, 499-507 (1999).
4. Minghetti, L. et al. J. Neuropathol. Exp. Neurol., 59, 866-871 (2000).
5. Hodgson, E. K., and Fridovich, I. Biochemistry, 14, 5294-5299 (1975).
6. Yim, M. B., Chock, P. B., and Stadtman, E. R. J. Biol. Chem., 268, 4099-4105 (1993).
7. Petyaev, I. M. in Superoxide Dismutase: Recent Advances and Clinical Applications (ed. Edeas, M. A.) 40-44 (Mel Paris, Paris-Tokyo, 1999).
8. Dearmond, S. J., and Prusiner S. B. in Greenfield's Neuropathology (eds Graham, D. I., and Lantos P. L.) 235-280 (Arnold, London-Sydney-Auckland, 1997).

TABLE 1

| | Cerebral cortex | | Cerebellum | |
|---|---|---|---|---|
| Activities | Grey matter | White matter | Grey matter | White matter |
| Control | | | | |
| SOD | 1.2 ± 0.14 | 0.2 ± 0.07 | 0.8 ± 0.09 | 0.2 ± 0.04 |
| peroxidase | 0.4 ± 0.50 | 1.2 ± 0.34 | 0.2 ± 0.21 | 1.2 ± 0.16 |
| catalase | 33 ± 2.7 | | 28 ± 3.1 | |
| MND | | | | |
| SOD | 1.2 ± 0.29 | 1.1 ± 0.27 | 0.4 ± 0.08 | 0.3 ± 0.45 |
| peroxidase | 0.3 ± 0.05 | 2.3 ± 0.21 | 0.8 ± 0.15 | 2.7 ± 0.97 |
| catalase | 25 ± 3.9 | | 22 ± 2.5 | |
| NvCJD | | | | |
| SOD | 0.2 ± 0.06 | 0.1 ± 0.09 | 0.4 ± 0.06 | 0.2 ± 0.03 |
| | p < 0.01 | p > 0.05 | p < 0.05 | p > 0.05 |
| peroxidase | 5.7 ± 0.42 | 3.4 ± 0.45 | 2.4 ± 0.21 | 1.5 ± 0.29 |
| | p < 0.001 | p > 0.05 | p < 0.01 | p > 0.05 |

| | | |
|---|---|---|
| catalase | 0 ± 2.6 | 8.1 ± 1.2 |
| | p < 0.001 | p < 0.01 |

1. A method for identifying and/or obtaining an agent for the treatment of spongiform encephalopathy comprising;
i) contacting a hydrogen peroxide degrading enzyme with a test compound; and,
ii) determining the activity of the hydrogen peroxide degrading enzyme.

2. A method according to claim 1 wherein the hydrogen peroxide degrading enzyme is comprised in a cell.

3. A method according to claim 1 wherein an increase in hydrogen peroxide degrading enzyme activity is indicative that the compound is candidate agent for the treatment of spongiform encephalopathy.

4. A method according to claim 2 wherein the cell is a human cell.

5. A method according to claim 2 wherein the cell is a neuron.

6. A method according to claim 5 wherein the cell is a brain neuron.

7. A method according to claim 1
wherein the hydrogen peroxide degrading enzyme is catalase or glutathione peroxidase.

8. A method according to claim 1 comprising determining the ability of said compound to cross the BBB.

9. A method for identifying and/or obtaining an agent for the treatment of spongiform encephalopathy comprising;
introducing a test compound into a non-human animal; and, determining the activity of a hydrogen peroxide degrading enzyme activity in the brain of said animal.

10. A method according to claim 1 comprising identifying a test compound as an agent which modulates the expression and/or activity of hydrogen peroxide degrading enzyme in said cell.

11. A method according to claim 10 comprising isolating and/or purifying a test compound identified as an agent which modulates the expression and/or activity of hydrogen peroxide degrading enzyme in said cell.

12. A method according to claim 10 comprising preparing and/or synthesising the test compound.

13. A method according to claim 11 comprising modifying the test compound.

14. A method according to claim 13 wherein the compound is modified to cross the BBB.

15. A method according to claim 14 wherein the compound is modified by lipidization.

16. A method according to claim 14 wherein the compound is modified by conjugation to an antibody molecule which binds to a BBB receptor.

17. A method according to claim 11 comprising formulating the test compound into a composition with a pharmaceutically acceptable excipient.

18. An agent which modulates the expression and/or activity of hydrogen peroxide degrading enzyme, said agent being identified by a method of claim 10.

19. A method of producing a pharmaceutical composition comprising;
identifying a compound which modulates the expression and/or activity of hydrogen peroxide degrading enzyme using a method according to claim 10,
admixing the compound identified thereby with a pharmaceutically acceptable carrier.

20. A method for preparing a pharmaceutical composition for treating a spongiform encephalopathy comprising;
i) identifying a compound which is a modulator of the expression and/or activity of hydrogen peroxide degrading enzyme,
ii) synthesising the identified compound, and;
iii) incorporating the compound into a pharmaceutical composition.

21. A pharmaceutical composition comprising an agent according to claim 18.

22. A method treating a spongiform encephalopathy comprising administration of a agent according to claim 18 to a patient for treatment of a spongiform encephalopathy.

23. Use of an agent according to claim 18 in the manufacture of a medicament for the treatment of a spongiform encephalopathy.

24. A method of making a pharmaceutical composition comprising admixing an agent according to claim 18 with a pharmaceutically acceptable excipient.

25. A method of treating a spongiform encephalopathy comprising; enhancing the expression and/or activity of hydrogen peroxide degrading enzyme in the brain of said individual.

26. A non-human animal model for use in screening for compounds useful in the treatment of spongiform encephalopathy said animal having reduced or deficient levels and/or activity of a hydrogen peroxide degrading enzyme.

27. A non-human animal model according to claim 26 wherein the animal displays one or more symptoms of spongiform encephalopathy.

28. A method of producing a non-human animal model for use in screening for compounds useful in the treatment of spongiform encephalopathy comprising;
reducing or abrogating the expression and/or activity of a hydrogen peroxide degrading enzyme in an animal.

29. Use of a non-human animal having reduced or deficient levels and/or activity of hydrogen peroxide degrading enzyme in a method of screening for a compound which is useful in the treatment of spongiform encephalopathy.

30. A method of determining the susceptibility of a non-human animal to a spongiform encephalopathy comprising;
determining the level or activity of hydrogen peroxide degrading enzyme in a sample obtained from said animal.

31. A method of reducing the susceptibility of a non-human animal to a spongiform encephalopathy comprising;
increasing the expression and/or activity of hydrogen peroxide degrading enzyme in the brain of said animal.

32. A method according to claim 31 comprising;
introducing a recombinant nucleic acid encoding a hydrogen peroxide degrading enzyme into one or more cells of said animal.

33. A method of producing a non-human animal having reduced spongiform encephalopathy susceptibility comprising:
a) introducing a nucleic acid encoding a hydrogen peroxide degrading enzyme into a fertilized egg or an embryo of an animal;
b) transferring the egg or embryo comprising the nucleic acid to a surrogate mother animal; and
c) allowing the transferred egg or embryo comprising the nucleic acid to develop to term to produce a transgenic animal whose genome comprises the nucleic acid.

34. A method according to claim 33 wherein the nucleic acid further comprises a regulatory sequence operably linked to the nucleic acid sequence encoding the hydrogen peroxide degrading enzyme.

35. A method according to claim 33 comprising providing progeny of the transgenic animal, the genome of the progeny comprising a heterologous nucleic acid encoding a hydrogen peroxide degrading enzyme.

36. A method for determining the inclusion of a non-human animal in a breeding program to reduce the susceptibility to spongiform encephalopathy in progeny comprising:
determining the level or activity of hydrogen peroxide degrading enzyme in a tissue sample from the animal,
enhanced or increased activity or levels being indicative of an inheritable trait of reduced susceptibility to spongiform encephalopathy; and,
selecting for inclusion in said breeding program animals that exhibit enhanced or increased levels of hydrogen peroxide degrading enzymes.

37. A non-human animal having reduced susceptibility to spongiform encephalopathy, said animal having reduced or deficient levels and/or activity of a hydrogen peroxide degrading enzyme.

38. A non-human animal having reduced susceptibility to spongiform encephalopathy, said animal having reduced or deficient levels and/or activity of a hydrogen peroxide degrading enzyme wherein said animal is obtainable or obtained by a method according to claim 32.
